(19) United States
(12) Patent Application Publication
(10) Pub. No.: US 2025/0331830 A1
(43) Pub. Date: Oct. 30, 2025

(54) IMMUNE HEALTH EVALUATION

(71) Applicant: 4Life Patents, LLC, Sandy, UT (US)

(72) Inventor: David Vollmer, South Jordan, UT (US)

(21) Appl. No.: 19/189,232

(22) Filed: Apr. 24, 2025

Related U.S. Application Data
(60) Provisional application No. 63/638,290, filed on Apr. 24, 2024.

(51) Int. Cl. A61B 10/00; G01N 33/543; G01N 33/68

(52) U.S. Cl. CPC A61B 10/0051; G01N 33/54388; G01N 33/6854

(57) ABSTRACT

A system includes an oral fluid collector, a buffer, a lateral flow assay, and a portable electronic device. The system may be used at a point of contact with an individual to quickly and accurately obtain information about the individual's immune health. For example, the system may be used to obtain information about the amount of IgA present in the individual's saliva. Methods of determining an individual's immune health are also disclosed.

### CROSS-REFERENCE TO RELATED APPLICATION

A claim for priority to the Apr. 24, 2024 filing date of U.S. Provisional Patent Application No. 63/638,290, titled IMMUNE HEALTH EVALUATION (“the '290 Provisional Application”) is hereby made. The entire disclosure of the '290 Provisional Application is hereby incorporated herein.

### TECHNICAL FIELD

This disclosure relates generally to systems and methods for determining the immune health of a subject and, more specifically, to the analysis of analytes in the saliva of a subject to obtain a quick and accurate indication of the subject's immune health.

### RELATED ART

Immunoglobulins, which play important roles in immunity, are present within saliva. In particular, the immunoglobulin IgA is typically found within saliva. Salivary IgA (sIgA) has long been believed to provide an indication of a subject's immune health.

In past studies identifying and quantifying sIgA, the enzyme-linked immunosorbent assay (ELISA) has been the traditional method of quantifying concentrations of sIgA. Salivary immunoglobulin A (sIgA) is an antibody found in saliva, tears, and the human gastrointestinal (GI) tract. It plays a crucial role in human immune system health by attaching itself to potential health threats and neutralizing their activity. IgA levels found in saliva can represent the immune system's status at a given time. However, ELISAs are expensive, time-consuming, and require sending samples to a third-party lab, all of which prevent individuals from quickly and regularly monitoring their sIgA levels.

### SUMMARY

Systems and methods of this disclosure may quickly provide an accurate indication of the immune health of a subject. Optionally, systems and methods of this disclosure may include identifying one or more nutritional supplements and, as a further option, a dosage of each nutritional supplement that may enable the subject to maintain or improve their immune health.

A system of this disclosure may include a saliva collection device, a buffer, a lateral flow assay, and a portable electronic device. The portable electronic device may comprise a smart phone, a tablet computer, or the like.

The saliva collection device, which may also be referred to as an “oral fluid collector” (OFC), may be used to collect a sample of saliva, which may potentially include one or more analytes, from an individual. The saliva collection device may comprise any suitable device for collecting saliva from an individual. Optionally, a configuration of the saliva collection device may facilitate the production of saliva by a subject's salivary glands.

The buffer is an aqueous solution. The buffer may be provided in a container that receives the saliva collection device. The buffer may be formulated to facilitate removal of saliva from the saliva collection device. The buffer may dilute the saliva without adversely affecting any of analyte that may be present in the saliva.

The lateral flow assay, which may also be referred to as a “lateral flow device” (LFD), includes a sample pad that receives a sample of saliva in the buffer, a conjugate pad that holds reagents, at least one test line including immobilized molecules, a control line, and a wick that draws the sample from the sample pad, across the conjugate pad, across each test line, and across the control line. The lateral flow assay may also include a housing that defines a receptacle for the sample and a window through which each test line and the control line may be viewed. As analyte in a sample is immobilized by the immobilizing molecules of a test line, a reagent may make the immobilized analyte visible. As the sample and reagent pass the control line, the control line also becomes visible.

The portable electronic device may be a portable electronic device used by an individual whose saliva is being analyzed or by a person administering the assay. The portable electronic device may include a camera that obtains an image of the results of the lateral flow assay, a processor, and memory that stores an app executable by the processor to enable the processor to evaluate the results of the lateral flow assay. The app may enable the processor to determine a level of the analyte present in the sample. The app may, based at least partially on the level of analyte present in the sample, determine an immune health of the subject. Optionally, the app may enable the processor to identify one or more nutritional supplements and possibly dosages of one or more nutritional supplements that will help an individual maintain or improve their immune health. Some nonlimiting examples of suitable electronic devices are smart phones and tablet computers.

A method for determining an immune health of a subject may include collecting a sample of the saliva from the subject. Collection of the sample may occur with a saliva collection device. Once the sample has been collected, a buffer may be added to it. The buffer may dilute the sample without adversely affecting any analyte present in the sample. Dilution of the sample may also facilitate its use in a lateral flow assay. The diluted sample is then introduced onto a sample pad of a lateral flow assay. A wick near an opposite end of the lateral flow from the sample pad may draw the sample across the lateral flow assay. As the sample travels across the lateral flow assay, it may travel across or through a conjugate pad, at least one test line, and a control line. Once the lateral flow assay is complete, an image of each test line and the control line may be obtained. The image may be obtained with a camera of a portable electronic device. An app executed by a processor of the portable electronic device may cause the processor to process the image to determine a level (e.g., concentration, etc.) of an analyte present in the sample. The level of the analyte present in the sample may be indicative of an immune health of the subject; thus, the processor may provide an indication of the subject's immune health. Based at least partially on the level of the analyte, the processor may optionally identify one or more nutritional supplements, as well as a dosage of each nutritional supplement, that may enable the subject to maintain or improve their immune health.

The systems and methods of this disclosure may enable an individual to obtain an indication of a subject's (e.g., their own, another person's, etc.) immune health in a manner of minutes without the requirement of laboratory equipment or specialized training. In addition, the systems and methods of this disclosure may provide an individual with recommendations on how to maintain or improve the immune health of the subject.

Other aspects of the disclosed subject matter, as well as features and advantages of various aspects of the disclosed subject matter, should be apparent to those of ordinary skill in the art upon considering the preceding disclosure, the description and figures that follow, and the appended claims.

### BRIEF DESCRIPTION OF THE DRAWINGS

In the drawings:

FIG. 1 is a schematic representation of an embodiment of a system that includes an oral fluid collector (OFC), a buffer, a lateral flow assay, and a portable electronic device;

FIGS. 2A and 2B illustrate use of the OFC of FIG. 1 to obtain a sample from a subject;

FIGS. 3A-3C schematically depict an embodiment of use of the buffer and lateral flow assay of FIG. 1 to detect the presence of an analyte in a sample;

FIG. 4 illustrates use of the portable electronic device of FIG. 1 to determine an amount of analyte present in the sample;

FIGS. 5A-5N depict an embodiment of a user interface with an app executed by the portable electronic device of FIG. 1 to facilitate a method of using the embodiment of the system of FIG. 1 to determine an amount of sIgA in a sample obtained from the subject; and

FIG. 6 is a graph showing a correlation of the data obtained using enzyme-linked immunosorbent assays (ELISAs) with data obtained using a cube reader, which is analogous to and provides substantially the same accuracy as the portable electronic device executing the app.

### DETAILED DESCRIPTION

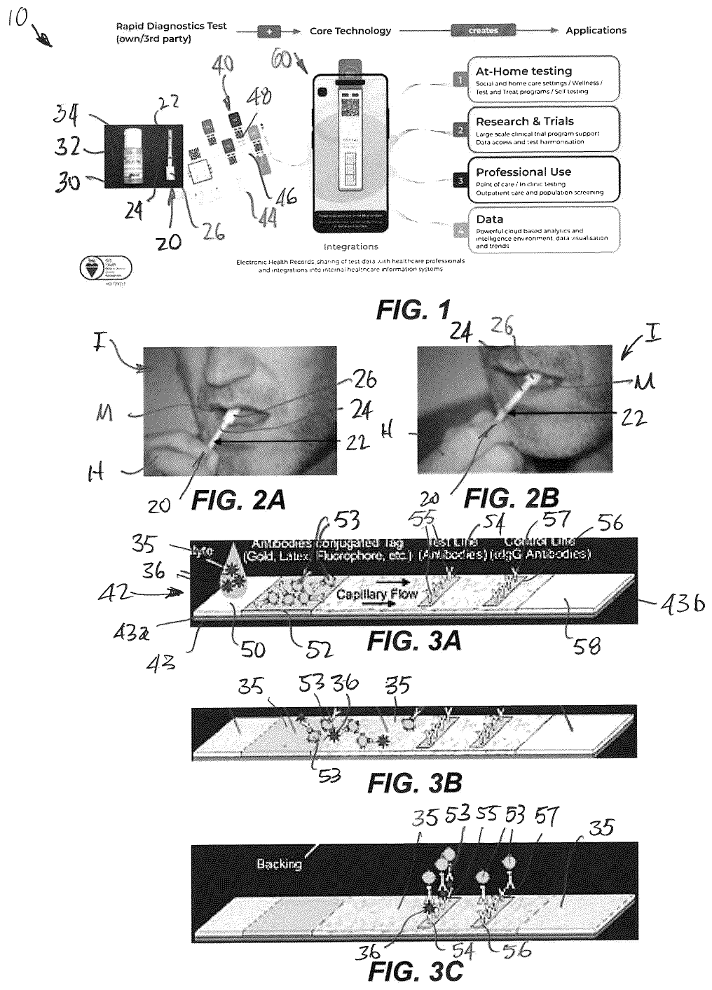

FIG. 1 is a schematic representation of an embodiment of a system 10 for quickly and accurately determining an amount of salivary IgA (sIgA) present in a subject's saliva, determining an immune state of the subject, and optionally providing the subject with recommendations on how to increase their immune state. Such a system 10 may include an oral fluid collector 20, a buffer 30, a lateral flow assay 40, and a portable electronic device 60. The oral fluid collector 20, buffer 30, and lateral flow assay 40 may be packaged together; i.e., in a kit. The portable electronic device 60 may be an individual's personal portable electronic device running an app that obtains results from a sample obtained with the oral fluid collector 20, diluted with the buffer 30, and applied to the lateral flow assay 40.

The oral fluid collector 20 may be used to collect a sample of saliva, which may potentially include one or more analytes, from an individual. The oral fluid collector 20 may comprise any suitable device for collecting saliva from an individual. Optionally, a configuration of the oral fluid collector 20 may facilitate the production of saliva by a subject's salivary glands.

The oral fluid collector 20 may be packaged in a container (e.g., a bag, etc.) (not shown). As illustrated, the oral fluid collector 20 may include a handle 22, an optional indicator 24, and a head 26.

The handle 22 may include a bottom end 21 and a top end 23. A shape and a length of the handle 22 may enable it to be easily grasped and manipulated with an individual's hand. For example, the handle 22 may comprise an elongated element, such as a plastic tube. The handle 22 may resemble a lollipop stick.

The optional indicator 24 may be positioned along a length of the handle 22; for example, at a somewhat intermediate location or adjacent to the top end 23 of the handle 22. The indicator 24 may comprise a porous material that can receive and retain a fluid, such as saliva. The indicator 24 may also include an indicator die 25 (FIG. 2B) that becomes visible when the indicator die 25 is exposed to a particular fluid, such as saliva, as may occur when the indicator 24 has been saturated with the fluid.

The head 26, which may be enlarged relative to the handle 22, may be located at the top end 23 of the handle 22. The head 26 may surround the top end 23 of the handle 22. The head 26 may comprise a porous material that collects and retains fluid. The porous material of the head 26 may be the same as the porous material of the optional indicator 24. The head 26 may be continuous with the optional indicator 24, such that a fluid received by the head 26 may be conveyed (e.g., by capillary action, etc.) to the indicator 24. A shape of the head 26 may facilitate the production of saliva by an individual's salivary glands when placed within the individual's mouth.

In a specific embodiment, the oral fluid collector 20 may comprise a swab.

FIGS. 2A and 2B depict an embodiment of a method of using the oral fluid collector 20 to collect a sample of saliva from the mouth M of an individual I. In such a method, the individual I may open a container that contains the oral fluid collector 20 and grasp the handle 22 with their hand H. Then, as shown in FIG. 2A, the individual I may place the head 26 of the oral fluid collector 20 in their mouth M. More specifically, the individual I may place the head 26 on top of their tongue T and close their mouth. The individual may leave the head 26 in place on top of their tongue T with their mouth closed as saliva from various salivary glands within the individual I's mouth pools on top of the tongue T. As the individual waits, they may resist the urge to suck saliva from the head 26. The individual I may leave the head 26 in place on top of their tongue T for at least 20 seconds (e.g., at least 30 seconds, at least 40 seconds, at least 45 seconds, at least 50 seconds, at least 60 seconds, etc.) or until the indicator 24 indicates that the head 26 has collected a sufficient quantity (i.e., volume) of saliva, as shown in FIG. 2B.

With returned reference to FIG. 1, the buffer 30 is an aqueous solution. The buffer 30 may be formulated to facilitate removal of saliva from the oral fluid collector 20. The buffer 30 may dilute the saliva without adversely affecting any of analyte that may be present in the saliva.

The buffer 30 may be provided in and contained by a container 32 (e.g., a bottle, etc.). The container 33 may have a shape and size that enable it to receive the oral fluid collector 20. The container 32 may receive a lid 34. The lid 34 may be removed from the container 32 or opened to provide access to the buffer 30 within the container 32. The lid 34 may be replaced on the container 32 or closed to enclose and optionally seal the buffer 30 and any other contents of the container 32 (e.g., the oral fluid collector 20, etc.) within the container 32.

Resuming the description of the embodiment of the method, once the head 26 of the oral fluid collector 20 has collected a sufficient quantity of saliva, the lid 34 of the container 32 for the buffer 30 may be removed from the container 32 or opened and the oral fluid collector 20 may be placed within the container 32. More specifically, the oral fluid collector 20 may be introduced head 26—first into the container 32 so that the head 26 may be fully immersed within the buffer 30 within the container 32. The lid 34 may then be replaced on the container 32 or closed to enclose the oral fluid collector 20 and buffer 30 within the container 32. The container 32 and its contents may then be agitated (e.g., moved with a rhythmic up-and-down motion, moved with a rhythmic back-and-forth motion, moved in a circular motion, etc.) to cause the buffer 30 to move around and into the head 26 of the oral fluid collector 20 so the buffer 30 can extract the saliva from the head 26. Such agitation may continue for a predetermined period of time (e.g., at least 30 seconds, at least 60 seconds, at least 90 seconds, at least 120 seconds, etc.).

The embodiment of the lateral flow assay 40 shown in FIG. 1 includes a substrate 42 that may be carried by a housing 44. The housing 44 includes a sample window 45 and at least one test window 46. The housing 44 may carry indicia 47 identifying the analyte that may be detected by the assay. The housing 44 may also carry a quick response (QR) code 48 that may enable the portable electronic device 60 to open and execute an app that corresponds to the lateral flow assay 40 and, optionally, to the particular analyte to be detected with the lateral flow assay 40.

Turning now to FIG. 3A, the substrate 42 of the lateral flow assay 40 may include a base 43, a sample pad 50, a conjugate pad 52, at least one test line 54, a control line 56, and an absorbent pad 58. A configuration of the substrate 42 may enable fluid to flow from the sample pad 50 to the absorbent pad 58.

The base 43 may comprise a strip with a first end 43a and a second end 43b. From the first end 43a to the second end 43b, the base 43 carries the sample pad 50, the conjugate pad 52, an assay pad that includes each test line 54 and the control line 56, and the absorbent pad 58. The base 43 may be formed from an inert material (e.g., a plastic, etc.).

The sample pad 50 is located adjacent to the first end 43a of the base 43. The sample pad 50 may be exposed through the sample window 45 (FIG. 1) of the housing 44 of the lateral flow assay 40. The sample pad 50 may be formed from a material that absorbs liquid (e.g., the sample 35, which includes saliva diluted by the buffer 30 (FIG. 1), etc.) and facilitates the transport of the liquid by capillary action, or wicking. More specifically, the material from which the sample pad 50 is formed may allow the liquid (e.g., the sample 35, etc.) to flow, by capillary action, along a length of the sample pad 50 and from the sample pad 50 to the conjugate pad 52. As an example, the sample pad 50 may comprise a porous matrix of a suitable material (e.g., cellulose, etc.).

The conjugate pad 52 is located on the base 43, adjacent to the sample pad 50, and on an opposite side of the sample pad 50 from the first end 43a of the base 43. The conjugate pad 52 holds a reagent 53. The reagent 53 may comprise antibodies that will bind to the analyte 36. The antibodies may be tagged with an indicator (e.g., a visible indicator, etc.). The conjugate pad 52 may be formed from a material that enables the reagent 53 to be carried by a liquid (e.g., the sample 35, etc.) and react with any molecules of analyte 36 present in the liquid. The material of the conjugate pad 52 may also facilitate the transport of the liquid along a length of the conjugate pad 52 to the assay pad. More specifically, the material from which the conjugate pad 52 is formed may enable the liquid to flow, by capillary action, along the length of the conjugate pad 52 and from the conjugate pad 52 to the assay pad, which includes each test line 54 and the control line 56. As an example, the conjugate pad 52 may comprise a porous matrix of a suitable material (e.g., glass fibers, etc.).

The assay pad, which includes each test line 54 and control line 56, is located on the base 43, adjacent to the conjugate pad 52, and on an opposite side of the conjugate pad 52 from the sample pad 50. The assay pad may be formed from a material that facilitates the transport of the liquid along a length of the assay pad, past each test line 54, past the control line 56, to the absorbent pad 58. More specifically, the material from which the assay pad is formed may enable the liquid to flow, by capillary action, along the length of the assay pad and from the assay pad to the absorbent pad 58. As an example, the assay pad may comprise a porous matrix of a suitable material (e.g., cellulose, etc.).

Each test line 54 and the control line 56 may be visible through the test window 46 (FIG. 1) of the housing 44 of the lateral flow assay 40.

Each test line 54 of the assay pad includes immobilized molecules 55 that provide a visible indication of analyte 36 present within a liquid (e.g., the sample 35, etc.). The immobilized molecules 55 may comprise antibodies that will bind to the analyte 36. As the liquid passes the immobilized molecules 55, the immobilized molecules 55 may bind molecules of analyte 36 that have already been bound by the reagent 53. Thus, the visible indicator of the reagent 53 may collect along the test line 54 to provide a visual representation of the presence of analyte 36 within the liquid and of an amount of the analyte 36 present within the liquid. For example, when low concentrations of the analyte 36 are present in the liquid, the test line 54 may appear faint, or light; with increasingly higher concentrations of analyte 36, the test line 54 may darken.

The control line 56 of the assay pad includes immobilized molecules 57 that provide a visible indication of that the liquid (e.g., the sample 35, etc.) has flowed along the length of the assay pad. The immobilized molecules 57 may comprise antibodies that bind to the antibodies of the conjugate 53. The extent to which the control line 56 darkens as the liquid flows past it may provide a reference for the amount of analyte 36 present in the liquid.

The absorbent pad 58 may be located adjacent to the assay pad, at the second end 43b of the base 43 of the substrate 42 of the lateral flow assay 40. The absorbent pad 58 may be formed from a material that collects the liquid that flows into the absorbent pad 58. More specifically, the material from which the absorbent pad 58 is formed may enable the liquid to flow, by capillary action, into the absorbent pad 58 to facilitate the transport of as=much of the liquid (e.g., the sample 35, etc.) as possible past the test line(s) 54 and control line 56. As an example, the absorbent pad 58 may comprise a porous matrix of a suitable material (e.g., cellulose, etc.).

The lateral flow assay of the system may comprise any suitable lateral flow assay. In a specific embodiment, the lateral flow assay 40 may be the IgA assay available from SOMA Bioscience.

Resuming the description of the embodiment of the method, as shown in FIG. 3A, a sample 35 of saliva that has been diluted in the buffer 30 (FIG. 1) is applied to the sample pad 50 of the substrate 42 of the lateral flow assay 40. As shown in FIG. 3B, the sample 35 flows from the sample pad 50 to the conjugate pad 52, where reagent 53 binds to analyte 36 (e.g., sIgA, etc.) present within the sample 35. As the sample 35 flows into the assay pad, reagent 53 that has bound to analyte 36 within the sample 35 is carried with the sample 35. Next, as shown in FIG. 3C, as the sample 35 flows past each test line 54, analyte 36 present in the sample 35 is bound by the immobilized molecules 55, holding the analyte 36, the bound reagent 53b, and the visible indicator of the bound reagent 53b along the test line 54. As the sample 35 flows past the control line 56, unbound reagent 53u present in the sample 35 is bound by the immobilized molecules 57, holding the previously unbound reagent 53u and the visible indicator of the previously unbound reagent 53u along the control line 56. In various embodiments, a color of the control line may differ from a color of each test line 54.

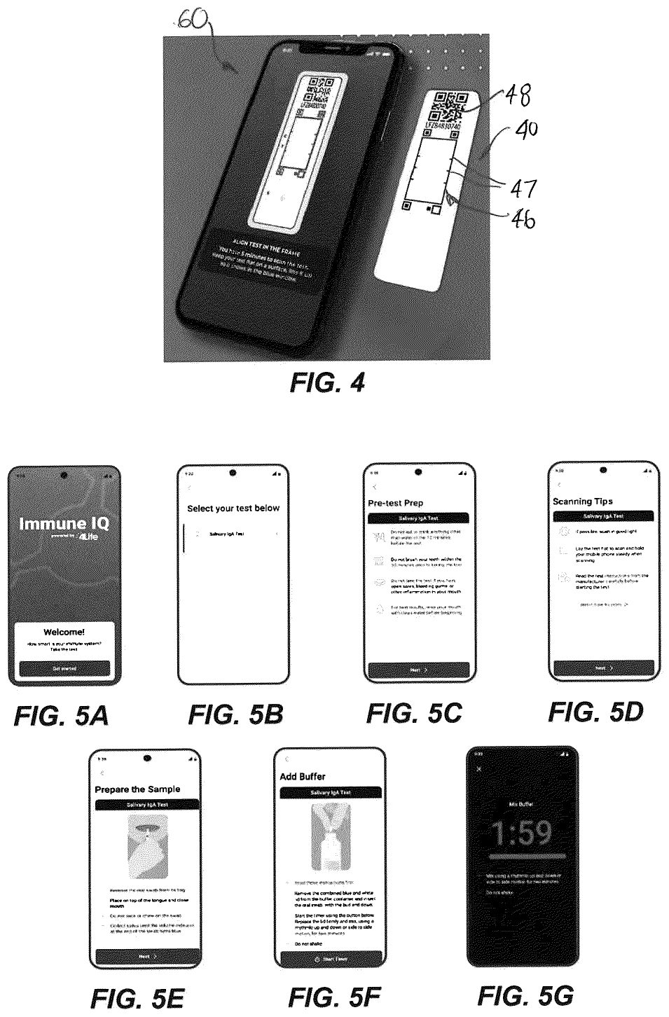

Turning now to FIG. 4, an embodiment of the portable electronic device 60 is illustrated. The portable electronic device 60 may be a portable electronic device used by an individual whose saliva is being analyzed or by a person administering the assay. The portable electronic device 60 may include a camera, a processor, and memory.

The camera of the portable electronic device 60 may be used to scan the QR code 48 on the housing 44 of the lateral flow assay 40, which may enable the processor of the portable electronic device 60 to execute an app. The app may be accessed from the internet or it may be stored by the memory of the portable electronic device 60.

The camera of the portable electronic device 60 may obtain an image of the results of a lateral flow assay 40, as shown in FIG. 4. When such an image is obtained by the camera of the portable electronic device 60, the app stored by the memory of the portable electronic device 60 and executed by the processor of the portable electronic device 60 may enable the processor to determine a level of analyte 36 (FIG. 3C) present in a sample 35 with which the lateral flow assay 40 was used.

The camera of the portable electronic device 60 may obtain an image of, or scan, the QR code 48 on the housing 44 of the lateral flow assay 40. The processor of the portable electronic device 60, under control of the app, may then automatically associate the specific lateral flow assay 40 identified by the QR code and the results obtained with the lateral flow assay 40 with a particular individual. For example, the identifying information embodied by the QR code 48 may automatically associate the results obtained with the lateral flow assay 40 with the individual who provided a sample for the lateral flow assay 40. As another example, the identifying information embodied by the QR code 48 may automatically associate the results obtained with the lateral flow assay 40 with the owner of the portable electronic device.

The processor of the portable electronic device 60 may, while executing the app, determine an immune health of the subject based at least partially on the level of analyte 36 present in the sample 35. The determination of immune health may be correlated and/or compared with any prior determination of the same individual's immune health. The determination of immune health may also be correlated and/or compared with similarly made determinations of the immune health of one or more other individuals. The other individuals may be part of a common group, such as a geographic group, a familial group, a demographic group (e.g., based on age, sex, race, physical fitness, health history, etc.).

Again resuming the description of the embodiment of the method, use of the portable electronic device 60 may include positioning the portable electronic device 60 over the lateral flow assay 40 with the camera of the portable electronic device 60 facing the lateral flow assay 40. The camera may be used to capture an image of the QR code 48 on the housing 44 of the lateral flow assay 40, which may automatically activate an app available through the internet or an app stored by memory of the portable electronic device 60.

As the processor of the portable electronic device executes the app, the app may provide various instructions, including instructions regarding previously described acts of the method. Once the lateral flow assay 40 is complete, the portable electronic device 60 may be positioned over the lateral flow assay 40 with the camera facing the test window 46 of the lateral flow assay 40. The camera, under control of the app, may then obtain an image of the test window of the lateral flow assay 40. The app may then evaluate that image (e.g., the test line 54, the control line 56, etc.), to determine how much of the analyte 36 was present in the sample (FIG. 3C).

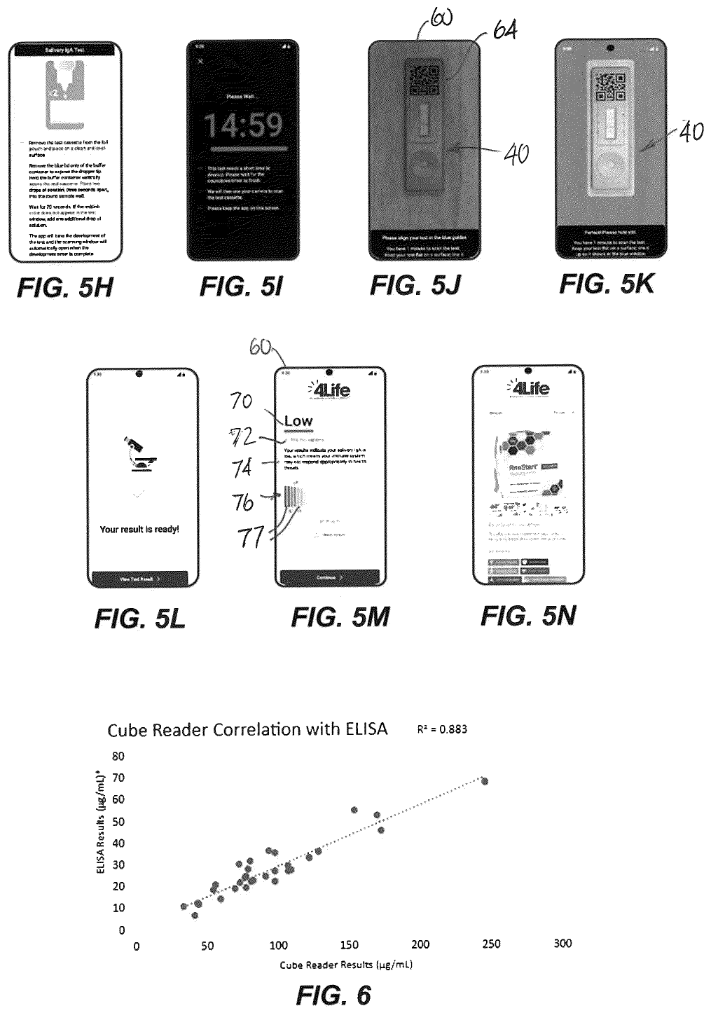

Turning now to FIGS. 5A-5N, a specific embodiment of an app is described.

FIG. 5A shows a welcome page of the app. The welcome page may included a button that may enable a user to cause the processor of the portable electronic device 60 (FIG. 4) to run, or execute, the app.

FIG. 5B is an embodiment of a page generated by the app that enables a user to select a type of test with which the app is to be used. As shown, the test may comprise a salivary IgA (sIgA) test. Once the user selects a test, the app may generate and display another page.

FIG. 5C depicts an embodiment of an instruction page generated by the app. The instruction page may provide a user with instructions on how to prepare for the test. By way of example, the instruction page may provide the user with instructions on various actions that must not be taken and/or various actions that must be taken before the test can be taken. The instruction page may also provide information on certain conditions that may adversely affect the accuracy of results obtained with the test. The instruction page may include a button that enables a user to continue to a subsequent page once the user understands the instructions for preparing for the test.

FIG. 5D depicts an embodiment of a tip page generated by the app. The tip page provides introductory information on how to use the portable electronic device 60 (FIG. 4) and its camera to obtain accurate results from with the test. The tip page may include links to further information, including test (e.g., lateral flow assay 40 (FIG. 1), etc.) instructions, instructional videos, and the like. Notably, such instruction may be provided before the user starts obtaining a sample to be used in the test. The timing of such instruction may ensure that the user is able to use the portable electronic device 60 to obtain accurate results once the test has been started. The tip page may include a button that enables a user to continue to a subsequent page once the user understands the instructions on how to use the portable electronic device 60 to obtain accurate results from the test.

FIG. 5E depicts an embodiment of a page generated by the app that provides a user with instructions on how to obtain a sample from an individual. For example, such a page may include instructions on how to use an oral fluid collector 20 (FIG. 1) to obtain a salivary sample from an individual, as described in reference to FIGS. 2A and 2B. Such a page may include a button that enables a user to continue to a subsequent page once the user obtains a sample from an individual (e.g., themselves or another individual).

FIG. 5F depicts an embodiment of a page generated by the app that provides a user with instructions on what to do with the sample once it has been obtained. For example, such a page may provide a user with instructions on how add the sample (e.g., the oral fluid collector 20 carrying the sample, etc.) to the buffer 30, as described in reference to FIG. 1. Such a page may include a button that enables a user to start a timer as soon as the sample has been added to the buffer 30.

FIG. 5G depicts an embodiment of a buffer timer page generated by the app. The buffer timer page may display a timer indicating the duration of time the sample (e.g., the oral fluid collector carrying the sample, etc.) is to remain in the buffer 30. The buffer timer page may also display instructions on actions that are to be taken or not to be taken while the sample remains in the buffer (e.g., mixing instructions, etc.). Once the timer has counted down to zero, the app may automatically proceed to a subsequent page.

FIG. 5H depicts an embodiment of an assay page generated by the app. The assay page may provide instructions on how to prepare and perform the lateral flow assay 40.

FIG. 5I depicts an embodiment of an assay timer page generated by the app. The assay timer page may display a timer indicating the duration of time remaining until the assay is complete. Once the timer counts down to zero, the app may automatically proceed to a subsequent page.

FIG. 5J depicts an embodiment of an image capture page generated by the app. The image capture page may provide a window showing an image captured by a camera of the portable electronic device 60. The window may include an outline 64 of the lateral flow assay 40 to aid in aligning and positioning the portable electronic device 60 and its camera to obtain an image of the lateral flow assay 40. When the lateral flow assay 40 is substantially aligned with and substantially fills the outline 64, the app may automatically continue to a subsequent image capture page.

FIG. 5K depicts an embodiment of the subsequent image capture page generated by the app. The subsequent image capture page may provide further instructions indicating that the app is about to cause the camera to obtain an image (i.e., take a picture) of the lateral flow assay 40. The subsequent image capture page may optionally provide additional instructions if the camera or lateral flow assay 40 is moved, if lighting conditions are suboptimal, or if too much time has passed since the assay timer shown in FIG. 5I counted down to zero. Once the camera obtains an acceptable image of the lateral flow assay 40, the subsequent image capture page may indicate as much to the user.

The app may then process information from the image (e.g., characteristics of the test line 54 (FIG. 3C) and the control line 56 (FIG. 3C), etc.). As the app processes information from the image, the app may compare the information obtained from the image to a database of comparable information to determine an immune heal of the individual.

FIG. 5L depicts an embodiment of a processing page generated by the app. The processing page may provide an indication of the status of the app's processing the information from the image. The processing page may provide an indication once processing is complete. The processing page may include a button that enables a user to select the results of the processing (and of the test). Once a user selects such a button, the app may continue to a subsequent page.

FIG. 5M depicts an embodiment of a results page generated by the app. The results page may provide a graphic indication of the results obtained from processing the information from the lateral flow assay 40 (FIG. 5K) captured by the camera of the portable electronic device 60. More specifically, the results page may provide a graphic indication of the immune heal of the individual who provided the sample. Without limitation, the graphic indication may include one or more of a general indicator 70 of the individual's immune health (e.g., low, normal, high, etc.), a numerical indicator 72 of the amount of analyte 36 (FIG. 3C) (e.g., sIgA, etc.) present in the individual's saliva (e.g., in micrograms, or μg), an explanation 74 of the individual's potential ability to respond to health threats based on the general indicator or the numerical indicator, and a graph 76 showing the amount of analyte 36 present in the individual's saliva. The graph 76, if any, may comprise a series of bars 77 that are illuminated based on the amount of analyte 36 present in the individual's saliva. As an example, the bars 77 may light up progressively from red (low amounts of analyte 36), to orange (medium amounts of analyte 36), to yellow (medium-high amounts of analyte 36), to green (high amounts of analyte 36), to blue (very high amounts of analyte 36). Optionally, the results page may include a button that, when selected, enables the app to share the information displayed by the results page. The results page may also include a button that enables a user to continue to a subsequent page once the user has finished viewing the results page.

FIG. 5N depicts an embodiment of a recommendations page generated by the app. The recommendations page may identify actions that may be taken by the individual to improve their immune health. For example, the recommendations page may identify one or more nutritional supplements and possibly dosages of one or more nutritional supplements that will help the individual maintain or improve their immune health. The recommendations page may provide links to product pages from which the recommended products may be purchased. The recommendations page may also provide information on activities (e.g., physical activity, etc.) that may help the individual improve their immune health. The recommendations that are made may be based on the results determined by the app, alone or with other information about the individual (e.g., information about their health, their geographic location, their family history, their demographic information, etc.).

Two studies were conducted to evaluate the feasibility of the system of this disclosure. The first study (“Study A”) focused on variability related to time of day of sample collection, fasted vs. fed states of participants, and test result accuracy. Study A had eight participants. The second study (“Study B”) evaluated variability from user error in sample collection, CR reading, and storage techniques. Study A had five participants. A third study (the “Salivary IgA Normal Range Evaluation”; “Study C”) was conducted separately to evaluate typical sIgA to provide a preliminary reference range and database for sIgA levels. Thirty-nine (39) participants were evaluated in Study C.

### Study A

Eight (8) healthy volunteers were recruited. Volunteers did not meet the exclusion criteria (presence of mouth infection). Participants were instructed to rinse their mouths prior to testing at all visits and refrain from eating or drinking, except water, for at least three (3) hours prior to fasted visits. If participants brushed their teeth, they were instructed to finish brushing at least 30 minutes before the visit. During sample collection, participants filled out a form to confirm compliance with instructions. Failure to comply would lead to exclusion of data recorded during that visit.

Participants attended six visits over three (3) days (i.e., two visits per day). Samples were collected in the late morning, just before lunch (fasted state), and in the early afternoon after participants ate (fed state). During the first visit (morning, fasted state), researchers collected three (3) samples of sIgA from each participant, five (5) minutes apart. In all subsequent visits, one (1) sIgA sample was collected from each participant.

During each visit, participants were given instructions on how to collect their own saliva sample using the oral fluid collector 20, as shown in FIGS. 2A and 2B. Instructions were included on the sample collection form and explained by the study coordinator during the visit. Participants removed the SOMA OFC swab from the bag, placed the swab in their mouth on top of the tongue, and closed their mouth (participants were instructed not to suck). Participants kept the swab in their mouth and continued to collect until the volume adequacy indicator on the swab turned royal blue in color. Collection was to stop immediately after the swab indicator turns blue. The instructions were taken directly from the manual provided by SOMA Biosciences.

After collection, participants gave samples taken during the visit to a study coordinator. After the fluid was collected, the swab was placed in the buffer 30 (FIG. 1), (SOMA Buffer solution) for two (2) minutes and mixed gently. In order to measure both morning and afternoon samples together, measurement of sIgA was performed at 5:00 PM at the end of each day. Study coordinators measured sIgA using the lateral flow assay 40 as shown in FIGS. 3A-3C, allowing the lateral flow assay 40 to develop for ten (10) minutes. According to the SOMA Biosciences' manual, a SOMA LFD (lateral flow device), an immunochromatographic strip test, is used to quantify the concentration of the target biomolecules. Two (2) to three (3) drops of the sample 35 (i.e., the buffer 30/saliva mixture) were placed on the sample pad 50 of the lateral flow assay 40. The sample flowed through the lateral flow assay 40, picking up dried conjugates 53 on the way. As the mixture flows, gold labelled anti secretory sIgA analytes 36 are captured in the test window 46 and show up as a red line. If sIgA molecules are in the mixture, they bind to gold labelled anti secretory IgA antibody and prevent them from binding to the test line. The more sIgA in the mixture, the less intense the line will appear. The concentration of sIgA is inversely proportional to the intensity of the red line. The lateral flow assay 40 was allowed to incubate for ten (10) minutes at room temperature.

Once the lateral flow assay 40 had incubated for ten (10) minutes, a cube reader was used to measure the intensity of the red line and convert that into a corresponding sIgA concentration (μg/ml or mcg/ml) in the original sample 35. The cube reader is analogous to and provides substantially the same accuracy as the portable electronic device 60 executing the app of this disclosure. The cube reader was calibrated to each lot of the lateral flow assay 40 lot prior to each test. Samples 35 were processed according to methods described in the SOMA Biosciences' manual. Results were recorded on a Microsoft® Excel® spreadsheet.

All the samples that were collected on the first day of the study were also sent to a third party, Salimetrics, in their buffer solution. The four samples taken on the first day of the study, in their respective buffer solutions, were placed in a 4° C. environment immediately after measurement and then sent to Salimetrics. Salimetrics analyzed each sample using an ELISA. ELISA results were compared to the previous reading of the same samples using the lateral flow assay 40 after 10 minutes of incubation and the cube reader. Only samples taken on Monday were sent to Salimetrics to be analyzed using ELISA, all other samples were analyzed using the cube reader method only. The limit of detection for the cube reader method is 25 μg/mL, and the limit of detection for the ELISA method is 2.5 μg/mL.

Cube reader precision and incubation time were also evaluated in Study A. Eight samples, one from each participant, taken during the first visit were measured three times one after the other using the same cube reader and same lateral flow assay 40. Incubation time was also evaluated using eight saliva samples, one from each participant taken during the first visit. The saliva sample was dropped into one lateral flow assay 40 and then measured after 10 minutes. The same lateral flow assay 40 was then measured 5-minutes later at the 15-minute incubation period.

### Study B

Five of the eight volunteers from Study A were recruited to participate in a second study examining the efficacy of different collection techniques. Three volunteers from study A elected not to participate in study B. Participants were required to attend one (1) hour long visit and were fasted for at least three (3) hours prior. Before the visit, volunteers were instructed to refrain from eating or drinking, except water, from breakfast until their visit. If participants brushed their teeth, they were asked to finish at least 30 minutes prior to the visit.

During the hour long visit, three (3) saliva samples were collected from each participant.

### CONTROL Sample 1 Procedure (Sample Immediate)

Participants removed the SOMA OFC swab from the bag, swab was placed in the mouth, on top of the tongue and mouth was closed (participants were instructed not to suck). Participants kept swab in their mouth and continued to collect until the volume adequacy indicator on the swab turned royal blue in color. Collection was to stop immediately after the swab indicator turns blue. Samples were then analyzed using the cube reader shortly after collection. This is the method recommended by SOMA for saliva collection.

### Sample 2 Procedure (Sample 1 Minute):

Participants removed the SOMA OFC swab from the bag, swab was placed in the mouth, on top of the tongue and mouth was closed (participants were instructed not to suck). Participants kept swab in their mouth and continued to collect until the volume adequacy indicator on the swab turned royal blue in color. Collection was to stop 1 minute after the swab indicator turns blue. Samples were then analyzed using the cube reader shortly after collection.

### Sample 3 Procedure (Sample Sucking)

Participants removed the SOMA OFC swab from the bag, swab was placed in the mouth, on top of the tongue and mouth was closed, participants were instructed to suck on the swab during collection. Participants kept the swab in their mouth and continued to collect until the volume adequacy indicator on the swab turned royal blue in color. Collection was to stop immediately after the swab indicator turns blue. Samples were then analyzed using the cube reader shortly after collection.

The order of sample collection was randomized for each participant. Participants waited 30 minutes in between collection of each sample. Each volunteer received a form to fill out during testing. This form asked them to report compliance with fasting instructions and record sample IDs as they performed the tests. Instructions were included on each form for the participant to follow. Each form was customized for each participant to ensure randomization of sample collection order.

Lateral flow assay 40 variability and buffer solution stability were also evaluated during Study B. Five samples, one taken from each participant, were measured three times using three different lateral flow assays 40. Saliva sample was dropped into each lateral flow assay 40 and then measured after the 10-minute incubation period. To determine stability of samples after storage, one sample taken from each participant during study B was measured once using one lateral flow assay 40, then stored at 4° C. for 9-10 days. After 9-10 days in the refrigerator, the buffer solution was warmed to room temperature and then measured once using one lateral flow assay 40.

### Study C-Salivary IgA Normal Range Evaluation

The normal range evaluation was conducted after studies A and B with 39 participants, none of whom participated in studies A or B. Participants with mouth infections were excluded from the study. Originally, there were 43 volunteers, four were excluded due to noncompliance. Healthy men and women were recruited per inclusion and exclusion criteria via invitation through their company email (4Life Research LLC and Biomedical Research Laboratory LLC). The invitation email contained instructions concerning the sample collection visit.

The purpose of this evaluation was to collect additional data points from a wider range of participants to estimate a “normal range” for salivary IgA. Collection protocol followed the instructions from the manual from SOMA Biosciences. Participants were to collect saliva in the manner intended by the manufacturer, no modifications to protocol were made in this evaluation. During the visit, each participant collected their own saliva by following instructions on the sample collection form and instructions given verbally by the study coordinators. Participants were asked a series of questions to determine if they complied with the following conditions, fasted for at least 3 hours, not brushed teeth for at least 30 minutes. Four participant samples were excluded due to noncompliance.

After volunteers filled out and followed instructions of the sample collection form, samples were given to the study coordinator. The sample and buffer solution were mixed gently for 2 minutes. Samples were left at room temperature and were analyzed in singlet within 1 hour of the sample collection. Samples were processed according to methods described in the SOMA Biosciences' manual. Results were recorded on a Microsoft® Excel® spreadsheet.

### Results

The target of many of the measurements taken across Study A and Study B was to evaluate the variability in measurements due to the precision of the instruments, the robustness of the methodology, and biological variance. Table 1 divides each measurement into one of these three categories.

### Study A

A one-sided t-test was used to analyze the difference between all fasted and fed measurements. It was found that there was a significant difference between average sIgA readings taken in fasted vs fed subjects, (p-value=0.0104).

All subjects were evaluated in both fasted and fed states. In a fasted state all 8 subjects had saliva collected over three five-minute intervals, except for subject 2, who only had two viable samples. Subjects' sIgA was also monitored across three days. Samples were collected both in the morning (fasted) and in the afternoon (fed). All three sets of data were analyzed using ANOVA (single factor) and none of the p-values indicated a significant difference between time intervals both inter- and intra-day regardless of subjects being fasted or fed.

Variability in the Cube Reader was evaluated across three (3) readings of the same lateral flow assay 40. Readings were taken one after the other, with no time delay (p-value=0.9999). Incubation time was also evaluated; there was a significant difference between average lateral flow assay 40 readings allowed to incubate for the recommended 10 minutes vs 15 minutes (p-value: 0.0002).

### Study B

Subjects were asked to collect samples according to manufacturer instructions and then again to track variability caused by noncompliance with directions. It was found that samples where the sample collection stopped one (1) minute after the collection swab indicator turns blue were not found to be statistically different from sample collection stopped immediately after the indicator turned blue (p-value: 0.2174). The same was determined for samples collected with the instructions to suck vs does not suck on the collection swab (p-value: 0.7705). Data was analyzed using a two-sided T-test.

Samples were obtained from the five subjects during Study B to determine variability across laminar flow devices (lateral flow assays 40) (p-value=0.9289). Both sets of data were analyzed using ANOVA (single factor) and neither set of data indicated a significant difference across readings.

The buffer solution for the collection swabs was evaluated for stability after storage at 4° C. It was found in the five samples evaluated, there was a significant difference in average readings taken between the day of sample collection and 9-10 days after (p-value=0.0241). Data was analyzed using a one-sided T-test.

Correlation with ELISA

The data was obtained from Study A. All samples were analyzed first by the cube reader method, and the second analysis was done by a third party (Salimetrics) using the ELISA method. The data was then plotted to determine correlation. The protocol for the Salimetrics ELISA method called for a further dilution of the buffer solution by a factor of five. Data points were plotted as seen in FIG. 1 and found to have a high correlation (R\hat{ }2=0.883).

### Study C-Salivary IgA Normal Range Evaluation

Participants of this evaluation provided samples that were plotted in FIG. 2 (n=35). 39 participants gave saliva for measurement. Four readings were excluded due to participants eating too close to the time of testing. The data provided a range of measurements for sIgA (5 Number Summary: Minimum: 25 μg/mL, Quartile Q1:61.5 μg/mL, Median: 113.1 μg/mL, Quartile Q3:153.2 μg/mL, Maximum: 330.0 μg/mL. Based on these data, the following general indicators of immune health are proposed for the following ranges of sIgA:
Low 25-60 μg/mL
Medium 60-110 μg/mL
Medium high 110-150 μg/mL
High 150-200 μg/mL
Very high 200+μg/mL

### Discussion

Rapid point of contact (POC) tests are becoming increasingly available and there are many current and future applications that can make it easier for individuals and medical professionals to monitor biomarkers with fast and non-invasive techniques. This preliminary data displays the level of reliability of the cube reader and, thus, of a portable electronic device 60 executing the app of this disclosure. Most importantly, this device has been shown to have a high correlation with the current ELISA method for quantifying sIgA. Similar results and findings have been published when comparing this methodology to ELISA.

The cube reader is reasonably precise for a rapid method. The use of a portable electronic device 60 executing an app of this disclosure is believed to be comparably precise. The lateral flow assays 40 used to detect sIgA have more variability between units, although the level of variability would still be acceptable in some circumstances provided the user understands the limitations of this method. This method would be practical in athletic training facilities or other health-conscious organizations.

Sample collection may be performed at a standardized time, which may aid in minimizing variation so the user can track general sIgA levels over time. Additionally, further recording and documenting a standard range for the portable electronic device 60-app method will help to better understand and interpret the results for individuals and clinicians.

### CONCLUSION

It was found that the cube reader is highly precise when measuring from the same lateral flow assay 40. Lateral flow assays 40 themselves have some variance across different units although, that variance is not significant.

As expected, when proper protocol is not followed, this method can produce less than accurate and less than precise results. The variability between average measurements examining proper incubation time and buffer stability was found to be statistically significant. Other variations in proper collection techniques were not found to have statistically different outcomes. However, it is still recommended that potential users of this method follow the instructions provided for the best results. Despite any potential variation due to user error, results could still aid individuals in self-monitoring sIgA levels over time.

The average measurements for the inter-day fasted vs fed samples are statistically different. This would indicate users should be consistent when testing over time to do so at the same time of day in a fasted state. This practice is recommended in collection instructions.

There is a positive correlation between the ELISA results and the readings from the Cube Reader Method. The ELISA method included further dilution resulting in a dilution factor of five which contributes to a numerical output lower than the cube reader method (by an average of 3.5× lower). Furthermore, previous data collected using the cube reader method validates the claim that the two methods are highly correlated. There is a difference between the ranges of each method due to differences in dilution factors. However, because they correlate relatively well, use of the cube reader method and the comparable portable electronic device 60-app method can give a good general snapshot of sIgA levels.

The range collected from the salivary IgA normal range evaluation provides a preliminary benchmark to compare IgA levels to what has been observed in other subjects. Because ranges and values vary across methods like ELISA it is important to establish an expected range users can reference when monitoring their own sIgA levels. Companies and clinicians that recommend the use of this technology should continue to monitor and record ranges of typical sIgA readings and present them to users for reference.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

What is claimed:

1. A system for determining immune health from saliva, comprising:
a saliva collection device used to collect a sample of saliva from an individual, the saliva potentially including an analyte;
a buffer that receives the sample from the saliva collection device;
a lateral flow assay including:
a sample pad that receives the sample;
a conjugate pad that holds reagents;
a test line including immobilized molecules;
a control line; and
a wick that draws the sample from the sample pad, across the conjugate pad, across the test line, and across the control line;
a portable electronic device with:
a camera that obtains an image of the test line and the control line; and
a processor executing an application that:
processes the image to determine a level of the analyte present in the sample; and
identifies a nutritional supplement appropriate for the level of the analyte present in the sample.

2. The system of claim 1, wherein the processor of the portable electronic device processes the image obtained with the camera of the portable electronic device to:
confirm that the control line is visible;
determine whether the test line of the lateral flow assay immobilized at least one analyte in the sample; and
determine an approximate concentration of the at least one analyte present in the sample.

3. The system of claim 2, wherein the processor of the portable electronic device processes the image obtained with the camera of the portable electronic device to determine the approximate concentration of the at least one analyte present in the sample by comparing a thickness and/or darkness of the test line to a thickness and/or darkness of the control line.

4. The system of claim 2, wherein the processor of the portable electronic device identifies the nutritional supplement appropriate for the approximate concentration of the at least one analyte.

5. The system of claim 4, wherein the processor of the portable electronic device also identifies a dosage of the nutritional supplement appropriate for the individual based on one or more physical characteristics of the individual and the approximate concentration of the at least one analyte.

6. The system of claim 1, wherein the test line of the lateral flow assay includes a first test line including first immobilized molecules that bind a first analyte and a second test line including second immobilized molecules that bind a second analyte.

7. The system of claim 1, wherein the analyte comprises a salivary immunoglobulin.

8. The system of claim 7, wherein the salivary immunoglobulin includes any salivary IgA present in the sample.

9. The system of claim 1, wherein the portable electronic device includes a smart phone or a tablet computer.

10. A method for analyzing saliva from an individual to determine an immune health of the individual, comprising:
collecting a sample of the saliva from the individual;
adding a buffer to the sample to provide a diluted sample;
introducing the diluted sample onto a sample pad of a lateral flow assay;
allowing the sample to travel across a conjugate pad, a test line, and a control line;
obtaining an image the test line and the control line with a portable electronic device; and
processing the image with an app executed by a processor of the portable electronic device to determine a level of an analyte indicative of the immune health of the individual present in the sample.

11. The method of claim 10, further comprising:
identifying a nutritional supplement appropriate for the level of the analyte present in the sample with the app executed by the processor of the portable electronic device.

12. The method of claim 11, further comprising:
identifying a dosage of the nutritional supplement appropriate for the individual based on one or more physical characteristics of the individual and the approximate concentration of the at least one analyte.

13. The method of claim 10, wherein determining the level of the analyte indicative of the immune health of the individual present in the sample comprises:
confirming that the control line of the lateral flow assay is visible;
determining whether the test line of the lateral flow assay immobilized at least one analyte in the sample; and
determining an approximate concentration of the at least one analyte present in the sample.

14. The method of claim 13, wherein the determining the approximate concentration comprises comparing a thickness and/or darkness of the test line to a thickness and/or darkness of the control line.

15. The method of claim 13, wherein determining whether the test line of the lateral flow assay immobilized at least one analyte comprises:
determining whether a first test line of the lateral flow assay immobilized a first analyte in the sample; and
determining whether a second test line of the lateral flow assay immobilized a second analyte in the sample.

16. The method of claim 10, wherein the analyte comprises a salivary immunoglobulin.

17. The method of claim 16, wherein the salivary immunoglobulin includes any salivary IgA present in the sample.
